(19) United States
(12) Patent Application Publication
(10) Pub. No.: US 2007/0010776 A1
(43) Pub. Date: Jan. 11, 2007

(54) Cosmetic bandage

(76) Inventors: James E. Price, Shawnee, KS (US); Timothy P. Philipp, Bridgeton, MO (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 10/568,131

(22) PCT Filed: Aug. 6, 2004
(86) PCT No.: PCT/US04/25579

§ 371 Date: Jul. 21, 2006

Related U.S. Application Data
(60) Provisional application No. 60/493,169, filed on Aug. 7, 2003.

(51) Int. Cl. A61F 15/00; A61F 13/00

(52) U.S. Cl. 602/54

(57) ABSTRACT

A cosmetic bandage (1) and method for its manufacture for external application to a wound, injury, blemish or other disfigurement of the human skin to help facilitate healing and for providing cosmetic benefits. The bandage (1) preferably includes a transparent backing layer (2) and an adhesive layer (5) applied to the first side (10) of the backing layer (2). An absorbent pad (3) is attached to a portion of the first side (10) of the backing layer (2). The topside (20) of the absorbent pad (3) is visible through the transparent backing layer (2). The top side (20) of the absorbent pad (3) is coated or embedded with one or more nontoxic pigment compositions which are derma or skin matching, having a melanin likeness in appearance so as to substantially match human skin tones and colors. A method for manufacturing a cosmetic bandage (1) is useful in the external application.

### CROSS-REFERENCE TO RELATED APPLICATIONS

This application is related to U.S. Provisional Patent Application No. 60/493,169 filed Aug. 7, 2003 from which priority is claimed.

### STATEMENT REGARDING FEDERALLY SPONSORED RESEARCH

Not Applicable.

### TECHNICAL FIELD

This invention relates to a novel bandage and the construction thereof for providing skin tone matching for conventional bandages. In particular, the present invention is directed to a low cost method of manufacturing in which the skin tone is applied to one side of an absorbent pad forming the bandage. The absorbent pad is attached to a transparent strip so that the bandage appears very close to the skin tone of the user.

### BACKGROUND ART

Adhesive bandages having skin tone properties are known in the art. However, skin tone bandages have not been widely accepted because their construction was complicated, and the techniques for skin matching were not adequate. Consequently, these various attempts to hide the bandage have met with only mixed success, and the desire for matching skin tone bandages remains unresolved. Included in prior art attempts are U.S. Pat. No. 2,905,174 ('174) in which a transparent bandage is imprinted along the backing with a plurality of visible flesh colored markings. U.S. Pat. No. 3,687,136 ('136) discloses a bandage with a transparent backing, a gauze dressing, and a piece of colored film there between to obscure the gauze dressing. While the '136 patent might work for its intended purpose, it does not provide adequate skin matching capabilities, and the method of construction is complicated and unsuitable for mass production. U.S. Pat. No. 4,161,176 provides a bandage with many layers releasably secured atop one another. Each layer is a different color. With this construction, the wearer peals off layers to select a desired color.

None of these attempts recognize that a solution to pigmentation could be obtained by imprinted in the gauze portion of the bandage itself, so that extremely simple, and low cost method of construction, is provide, that construction being one in which a variety of skin tones can be present to a user.

### SUMMARY OF THE INVENTION

In accordance with this invention, generally stated, a cosmetic bandage is provided which includes a transparent backing layer having a top side and a bottom side. A transparent adhesive layer is applied to the bottom side of the backing layer. An absorbent pad having a top side and a bottom side is arranged so that one of the sides has a non-toxic pigment composition applied to it so as to be derma matching and having a melanin likeness and appearance. The composition preferably is applied by flood printing or a silk screening process, in which a dot matrix arrangement is employed to provide improved skin matching capabilities. In the preferred embodiment, the absorbent pad it self provides the dot matrix arrangement. In other embodiments, the dot matrix is provided by the printing method employed for transferring the pigment to the pad. The pigmented side of the pad is attached to the backing layer and is intended to be visible therethrough. The adhesive bandages of the present invention aid in the healing of external wounds or injuries and provide a cosmetic concealment of the wound or injury by color matching and pattern matching the appearance of the human skin or derma.

### BRIEF DESCRIPTION OF DRAWINGS

The objects of the invention are achieved as set forth in the illustrative embodiments shown in the drawings, which form a part of the specification.

FIG. 1 is an exploded view of one illustrative embodiment of bandage of this invention;

FIG. 2 is a diagrammatic view of a first method of obtaining color matching;

FIG. 3 is a diagrammatic view of a second method of obtaining color matching;

FIG. 4 is a diagrammatic view of a third method of obtaining color matching;

FIG. 5 is a diagrammatic view of a forth method of obtaining color matching;

FIG. 6 is a diagrammatic view of one illustrative process for manufacturing the bandage of the present invention;

FIG. 7 is a view in perspective of the cosmetic bandage of the present invention in applicational use.

FIG. 8 is a top plane view of the bandage shown in FIG. 1; and

FIG. 9 is a bottom plane view of the bandage shown in FIG. 1.

Corresponding reference characters indicate corresponding parts throughout the several views of the drawings.

### BEST MODE FOR CARRYING OUT THE INVENTION

The following detailed description illustrates the invention by way of example and not by way of limitation. This description will clearly enable one skilled in the art to make and use the invention, and describes several embodiments, adaptations, variations, alternatives and uses of the invention, including what I presently believe is the best mode of carrying out the invention. As various changes could be made in the above constructions without departing from the scope of the invention, it is intended that all matter contained in the above description or shown in the accompanying drawings shall be interpreted as illustrative and not in a limiting sense.

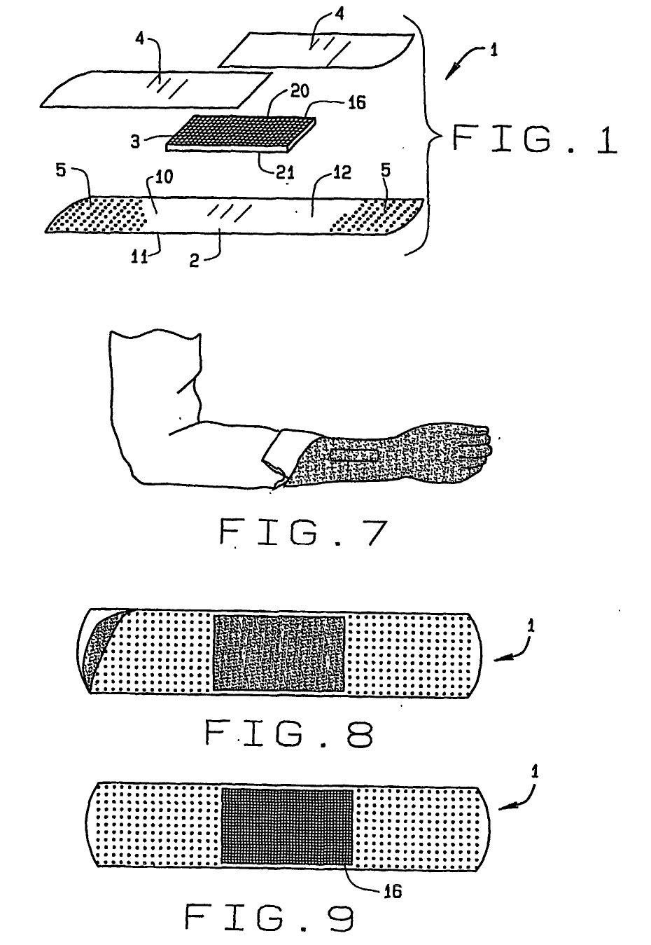

Referring now to FIG. 1, reference numeral 1 indicates one illustrative embodiment of the bandage of the present invention. Bandage 1 includes a backing layer 2, an absorbent pad 3, and at least one, and preferably two, removable strips 4.

The backing layer 2 includes a first side 10 and a second side 12. One of the sides 10 and 12 has a suitable adhesive 5 applied to it. In the preferred embodiment, the adhesive is pressure sensitive, but any suitable adhesive is compatible with the broader aspects of the present invention. The backing layer 2 preferably is transparent tape.

As will be appreciated by those skilled in the art, the backing layer 2 may assume a number of design shapes, and the silhouette of the backing layer maybe altered to conform to the desired shape.

The pad 3 preferably has a pattern construction 16 associated with it, best shown in FIGS. 1 and 9. The pattern structure is important in the construction of the bandage of this invention, in that it provides an effective dot matrix pattern, which enables one to construct the bandage of the present invention economically and quickly. The pad 3 also includes a top side 20 and a bottom side 21. The top side 21 is the side on which a pigment is applied as latter described in greater detail, while the bottom side 20 is the wound facing side of the bandage 1. Conventional removable strips 4 are provided to protect the bandage 1 prior to use.

As indicated, the pad 3 preferably has the matrix structure 16 associated with it, which permits the pad to have the pigment flood printed or silk screened on the pad. Preferably, the pigment maybe applied in a single step. In the alternative, a number of printing steps may be employed with one or more pigment tones or shades, to provide the proper color for the bandage. The printed side 21 of the pad 3 is then attached to the side 10 of the backing layer 2, so that the pigmented side of the pad 3 appears through the transparent backing 2. As will be appreciated by those skilled in the art, other printing or application processes are compatible with the broader aspects of our invention.

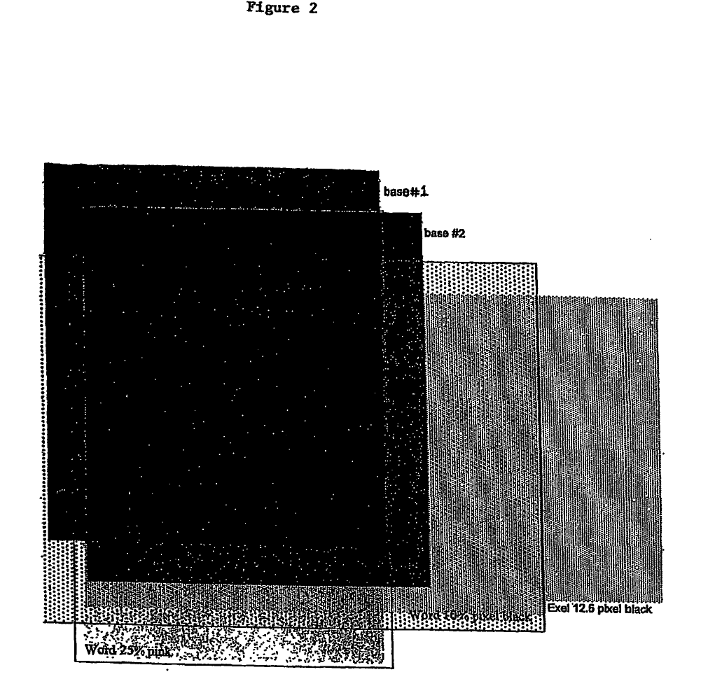

Alternatively, when the pad 3 is not formed with the dot matrix structure 20 initially, a plurality of these steps may be employed to provide the appearance of the pigmented layer. FIGS. 2-5 illustrate methods of obtaining the desired shading when the design does not include the matrix pattern 20.

As shown in FIG. 2, a first base color, such as a light flesh tone, is printed on the pad 3. Next, a second layer, such as a Microsoft Word® 10% pixel black, is printed onto the pad over the base color.

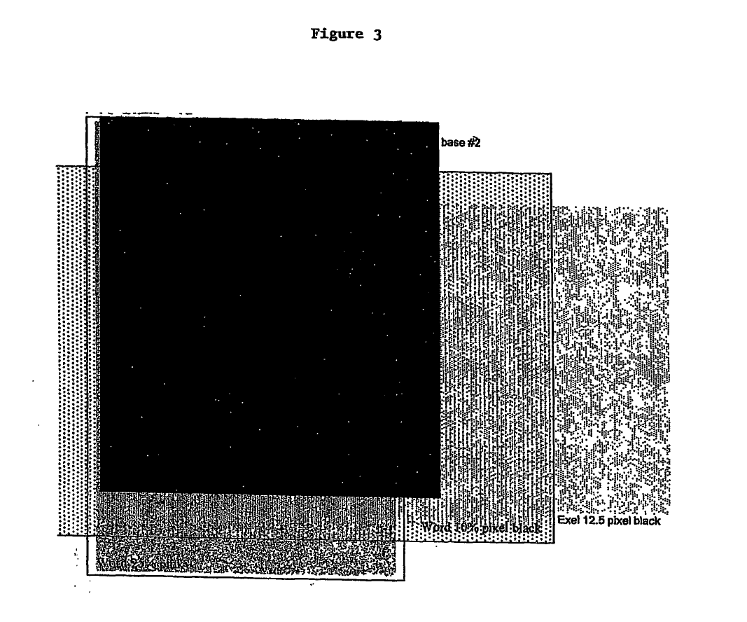

As shown in FIG. 3, a second base color, such as a light flesh tone, is printed on the pad 3. Next, a second layer, such as a Microsoft Word® 25% pink, is printed onto the pad over the base color. Then, a third layer, such as a Microsoft Excel® 12.5% pixel pink, is printed onto the pad over the second layer. Finally, a fourth layer, such as a Microsoft Word® 10% pixel black, is printed onto the pad over the third layer.

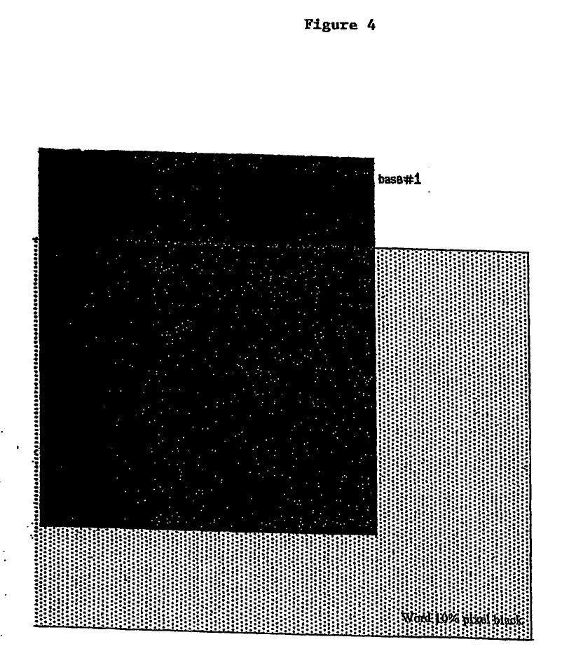

As shown in FIG. 4, a first base color, such as a light flesh tone, is printed on the pad 3. After that, a second base color, such as a light flesh tone, is printed on the pad over the first base color. Next, a third layer, such as a Microsoft Word® 25% pink, is printed onto the pad over the second base color. Then, a fourth layer, such as a Microsoft Excel® 12.5% pixel pink, is printed onto the pad over the third layer. Finally, a fifth layer, such as a Microsoft Word® 10% pixel black, is printed onto the pad over the third layer.

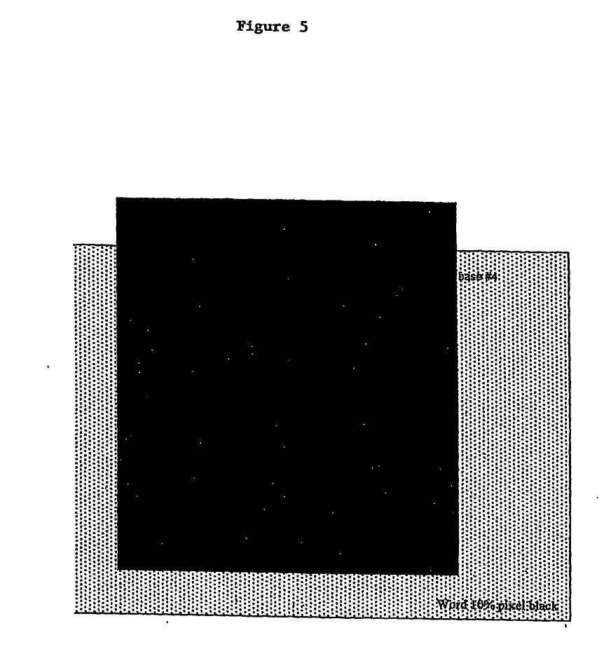

As shown in FIG. 5, a fourth base color, such as a light flesh tone, is printed on the pad 3. Next, a second layer, such as a Microsoft Word® 10% pixel black, is printed onto the pad over the fourth base color.

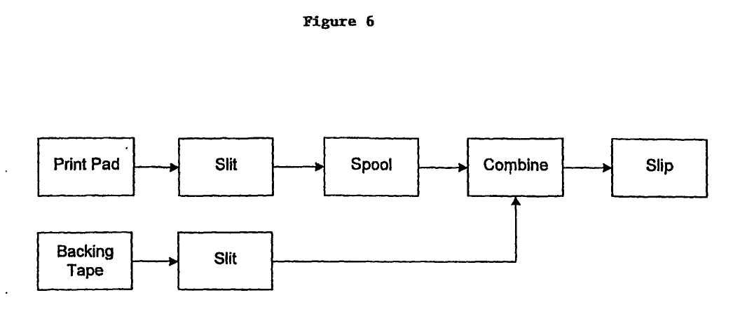

FIG. 6 illustrates one method of forming the bandage of the present invention. As there shown, the pad 3 material is first printed with a suitable pigment. As indicated, the printing may be a single step, or multiple printing steps may be used to achieve the desired color combination. Thereafter, the pad 3 is slit or cut and re-spooled. The backing tape 2 likewise is cut or slit to a desired configuration, and the pad 3 and tape 2 are attached to one another in a combining step. Thereafter, the removable backing strips 4 are applied to complete the bandage construction.

Numerous variations will occur to those skilled in the art in view of the forgoing description of the company drawings. As indicated, the shape and design silhouette of the bandage 1 may vary in other embodiments of this invention. Likewise, while certain techniques for applying the pigment to the pad for described, other methods are compatible with the broader aspects of this invention. The adhesive used in connection with the bandage may change in other embodiments of the invention. These variations are merely illustrative.

In view of the above, it will be seen that the several objects and advantages of the present invention have been achieved and other advantageous results have been obtained.

1. A cosmetic bandage comprising:
a transparent backing layer having a top side and a bottom side;
a transparent adhesive layer applied to the bottom side of the transparent backing layer;
an absorbent pad having a bottom side and a top side, the top having a non-toxic pigmented composition applied to it so as to be derma matching and having a melanin likeness and appearance so as to substantially match human skin tones and colors, the top side of the absorbent pad being attached to the bottom side of said backing layer so as to be visible therethrough; and
at least one removable strip applied to the bottom side of said backing layer and said pad.

2. The bandage of claim 1 wherein the adhesive is pressure sensitive.

3. The bandage of claim 2 further including a plurality of removable strips.

4. The bandage of claim 3 wherein the pigmented portion comprises a plurality of pigments blended to provide skin matching capability.

5. The bandage of claim 3 wherein the pigments are imbedded in the pad.

6. The bandage of claim 3 wherein the pigments are coated on the pad.

7. A method of constructing a cosmetic bandage comprising:
forming an absorbent pad having a bottom side and a top side;
applying a non-toxic pigment to the top side of the pad;
and attaching the pigmented top side of the pad to a transparent backing layer.

8. The method of claim 7 further including the step of applying an adhesive to the backing layer prior to attaching the pad.

9. The method of claim 8 including the step of providing a removable and disposable strip over the adhesive layer.

10. The method of claim 9 including the step of applying the pigment to the pad by a silk screening process.

11. The method of claim 9 including the steps of applying the pigment by a flood printing process.
